(19) United States
(12) Patent Application Publication
(10) Pub. No.: US 2026/0053446 A1
(43) Pub. Date: Feb. 26, 2026

(54) METHODS AND SYSTEMS FOR PREDICTING HEALTH RISKS

(71) Applicant: The United States Government as Represented by the Department of Veterans Affairs, Washington, DC (US)

(72) Inventor: Diane C. Lim, Washington, DC (US)

(21) Appl. No.: 19/109,022

(22) PCT Filed: Sep. 11, 2023
(86) PCT No.: PCT/US2023/032400

§ 371 Date: Mar. 5, 2025

Related U.S. Application Data
(60) Provisional application No. 63/405,109, filed on Sep. 9, 2022.

(51) Int. Cl. A61B 5/00; A61B 5/08; A61B 5/1455

(52) U.S. Cl. CPC A61B 5/7282; A61B 5/08; A61B 5/14551; A61B 5/7264

(57) ABSTRACT

Methods, systems, and apparatuses for determining a health metric and one or more health risks based on one or more biological events. Data indicative of one or more biological events may be determined based on a biological signal associated with an individual and a weighted factor may be determined for each biological event. The weighted factor may be applied to each biological event to determine one or more weighted biological events. A health metric may be determined based on the one or more weighted biological events. The health metric may be used to determine one or more health risks associated with the individual, wherein the individual may be treated based on the one or more health risks.

### CROSS REFERENCE TO RELATED PATENT APPLICATION

This application is a national phase filing under 35 U.S.C. § 371 of International Application No. PCT/US2023/032400, filed Sep. 11, 2023, which claims priority to U.S. Provisional Application No. 63/405,109, filed Sep. 9, 2022, which is herein incorporated by reference in their entirety.

### BACKGROUND

Oxygen saturation of peripheral blood (SpO₂), as measured by pulse oximetry, has been shown to have broad clinical utility, from neonates to adults, and from intensive care unit (ICU) devices to wearable devices. Hypoxemia is significant in numerous scenarios from sedation-related to exercise-induced, from rest during wake to nocturnal hypoxemia. However, there is a lack of a standard method of quantifying hypoxemia. Moreover, there are no standard methods for comparing the consequences of hypoxemia across different clinical contexts. For example, Sedation-related hypoxemia may occur in 1.8% to 69% of patients undergoing gastrointestinal endoscopic surgery. Sedation-related hypoxemia has been associated with adverse cardiac events and is the cause of 33% of malpractice claims. Postoperative hypoxemia may occur in 5% to 65.5% of patients and has been associated with prolonged hospital stay and increased mortality. Exercise induced hypoxemia (EIH) has been associated with severe chronic pulmonary diseases (COPD). In patients with fibrotic interstitial lung disease, EIH can be used to classify mortality and transplantation predictions. In COPD patients, EIH has been used to determine who should receive life-saving supplemental oxygen therapy. In endurance athletes, EIH has been used to classify and integrate altitude/hypoxic training. Resting hypoxemia has been used to facilitate decision-making processes in coronavirus disease 2019 (COVID-19) patients. Different methods of measuring hypoxemia severity have been developed to triage which patients should be admitted to the ICU versus outpatient management, to identify the predictive factors of severe hypoxemia, to understand the danger of “happy hypoxemia” (e.g., severe hypoxemia where patients are not dyspneic but can rapidly deteriorate), and to predict in-hospital mortality. Nocturnal hypoxemia is characterized by low SpO₂ levels during sleep, and is one component of diagnosing Obstructive Sleep Apnea (OSA) in children and in adults. The apnea-hypopnea index (AHI) is the current metric to diagnose OSA. The AHI measures nocturnal hypoxemia, such as the total sleep time under SpO₂ of 90% (TST90) or minimum saturation (Min Sat), and correlates to outcomes such as cancer incidence and mortality, atrial fibrillation, sudden cardiac death, and major adverse cardio vascular events (MACE). When OSA is treated with positive airway pressure (PAP), which normalizes oxygenation, there is an improvement in many, but not all outcomes. This is due to the fact that while TST90 quantifies time spent below 90%, it does not reflect the severity of hypoxemia during sleep. Moreover, although Min Sat is the lowest nadir of oxygen saturation during sleep, it does not reflect the temporal dimension of hypoxemia during sleep.

### SUMMARY

It is to be understood that both the following general description and the following detailed description are exemplary and explanatory only and are not restrictive.

Methods, systems, and apparatuses for an improved health metric determination and determinations of one or more health risks based on one or more biological events. Data indicative of one or more biological events may be determined based on a biological signal associated with an individual. A beginning of each event may be determined when the biological signal crosses from above to blow an upper threshold and an end of each event may be determined when the biological signal crosses from below to above the upper threshold. An area under the curve (AUC) and/or an area above the curve (AAC), below the upper threshold, may be calculated for each biological event. A weighted factor may be determined for each biological event. The AUC or the AAC of each biological event may be multiplied by the associated weighted factor to determine a weighted biological event. The health metric may be determined based on a sum of the weighted biological events multiplied by a normalization factor. One or more health risks associated with the individual may be determined based on the health metric.

In an embodiment, disclosed are methods comprising receiving, by a computing device, a biological signal associated with an individual, determining, based on the biological signal, data indicative of one or more biological events, wherein each biological event of the one or more biological events is associated with a weighted factor, determining, based on an application of each weighted factor to each biological event, one or more weighted biological events, and determining, based on the one or more weighted biological events, a health metric.

In an embodiment, disclosed are apparatuses comprising one or more processors, a memory storing processor-executable instructions that, when executed by the one or more processors, cause the apparatus to, receive a biological signal associated with an individual, determine, based on the biological signal, data indicative of one or more biological events, wherein each biological event of the one or more biological events is associated with a weighted factor, determine, based on an application of each weighted factor to each biological event, one or more weighted biological events, and determine, based on the one or more weighted biological events, a health metric.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of the present description serve to explain the principles of the methods and systems described herein:

FIG. 1 shows an example system;

FIG. 2 shows an example system environment;

FIG. 3 shows an example weighted hypoxemia index formulation;

FIGS. 4A-4C show example predicted survival curves;

FIG. 5 shows an example machine learning system;

FIG. 6 shows a flowchart of an example machine learning method; and

FIG. 7 shows a flowchart of an example method.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. Similarly, when values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, the methods and systems may take the form of a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

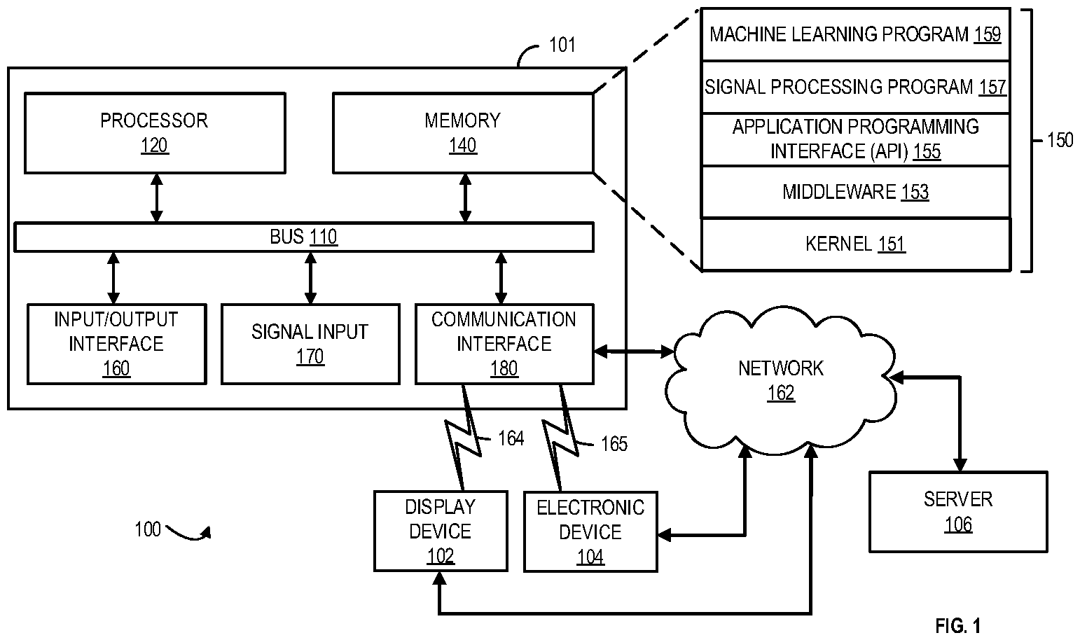

FIG. 1 shows an example system 100 for determining a health metric (e.g., weighted hypoxemia index) and one or more health risks based on one or more biological events. For example, a device (e.g., computing device 101) may receive a biological signal associated with an individual. Data indicative of one or more biological events may be determined based on the biological signal. A beginning of each biological event may be determined when the biological signal crosses from above to blow an upper threshold and an end of each event may be determined when the biological signal crosses from below to above the upper threshold. A weighted factor may be determined for each biological event. One or more of an area under the biological signal or an area above the biological signal below the upper threshold may be determined for each biological event. Either area associated with each biological event may be multiplied by the associated weighted factor to determine a weighted biological event. The health metric may be determined based on a sum of the weighted biological events multiplied by a normalization factor. One or more health risks associated with the individual may be determined based on the health metric. The system 100 may include a computing device 101, a display device 102, an electronic device 104, and one or more servers 106. In an example, the computing device 101 may be configured to receive the biological signal associated with the individual. In an example, the computing device 101 may be in communication with the display device 102, the electronic device 104, and the one or more servers 106 via a network (e.g., network 162).

The computing device 101 may include a bus 110, one or more processors 120, a memory 140, an input/output interface 160, a signal input 170, and a communication interface 180. In certain examples, the computing device 101 may omit at least one of the aforementioned elements or may additionally include other elements. The computing device 101 may comprise, for example, a laptop computer, a mobile phone, a smart phone, a tablet computer, a wearable device, a smartwatch, a haptic device, a desktop computer, a smart television, and the like.

The bus 110 may comprise a circuit for connecting the bus 110, the one or more processors 120, the memory 140, the input/output interface 160, the signal input 170, and/or the communication interface 180 to each other and for delivering communication (e.g., a control message and/or data) between the bus 110, the one or more processors 120, the memory 140, the input/output interface 160, the signal input 170, and/or the communication interface 180.

The one or more processors 120 may include one or more of a Central Processing Unit (CPU), an Application Processor (AP), or a Communication Processor (CP). The one or more processors 120 may control, for example, at least one of the bus 110, the memory 140, the input/output interface 160, the signal input 170, and/or the communication interface 180 of the computing device 101 and/or may execute an arithmetic operation or data processing for communication. As an example, the one or more processors 120 may drive (e.g., cause) the signal input 170 to receive/capture a pulse oximetry signal of an individual. The processing (or controlling) operation of the one or more processors 120 according to various embodiments is described in detail with reference to the following drawings.

The processor-executable instructions executed by the one or more processors 120 may be stored and/or maintained by the memory 140. The memory 140 may include a volatile and/or non-volatile memory. The memory 140 may include random-access memory (RAM), flash memory, solid state or inertial disks, or any combination thereof. As an example, the memory 140 may include an Embedded MultiMedia Card (eMMC). The memory 140 may store, for example, a command or data related to at least one of the bus 110, the one or more processors 120, the memory 140, the input/output interface 160, the signal input 170, and/or the communication interface 180 of the data capture device 101. According to various examples, the memory 140 may store software and/or a program 150 or may comprise firmware. For example, the program 150 may include a kernel 151, a middleware 153, an Application Programming Interface (API) 155, a signal processing program 157, and/or machine learning program/model 159, and/or the like, configured for controlling one or more functions of the data capture device 101 and/or an external device (e.g., the display device 102 or electronic device 104). At least one part of the kernel 151, middleware 153, or API 155 may be referred to as an Operating System (OS). The memory 140 may include a computer-readable recording medium (e.g., a non-transitory computer-readable medium) having a program recorded therein to perform the methods according to various embodiments by the one or more processors 120. In an example, the memory 140 may store the scans received from the pulse oximetry input 170.

The kernel 151 may control or manage, for example, system resources (e.g., the bus 110, the one or more processors 120, the memory 140, etc.) used to execute an operation or function implemented in other programs (e.g., the middleware 153, the API 155, the signal processing program 157, or the machine learning program/model 159). Further, the kernel 151 may provide an interface capable of controlling or managing the system resources by accessing individual elements of the data capture device 101 in the middleware 153, the API 155, the signal processing program 157, or the machine learning program/model 159.

The middleware 153 may perform, for example, a mediation role, so that the API 155, the signal processing program 157, and/or the machine learning program/model 159 can communicate with the kernel 151 to exchange data. Further, the middleware 153 may handle one or more task requests received from the signal processing program 157 and/or the machine learning program/model 159 according to a priority. For example, the middleware 153 may assign a priority of using the system resources (e.g., the bus 110, the one or more processors 120, or the memory 140) of the computing device 101 to at least one of the signal processing program 157 and/or the machine learning program/model 159. For example, the middleware 153 may process the one or more task requests according to the priority assigned to at least one of the application programs, and thus, may perform scheduling or load balancing on the one or more task requests.

The API 155 may include at least one interface or function (e.g., instruction), for example, for file control, window control, video processing, and/or character control, as an interface capable of controlling a function provided by the scan processing program 157 and/or the machine learning program/model 159 in the kernel 151 or the middleware 153.

As an example, the signal processing program 157 and the machine learning program/model 159 may be independent of each other or integrally combined, in whole or in part.

The signal processing program 157 may include logic (e.g., hardware, software, firmware, etc.) that may be implemented to cause the computing device 101 to process the biological signals received/taken by the signal input 170. As an example, the signal input 170 may comprise a sensor device configured to receive/capture a biological signal of an individual. For example, the biological signal may comprise one or more of a pulse oximetry signal, a CO₂ signal, an arterial catheter signal (e.g., radial, carotid, or femoral), a central venous catheter signal (e.g., pulmonary artery catheter, and the like), and the like. The signal processing program 157 may cause the computing device 101 to determine a health metric (e.g., weighted hypoxemia index) associated with an individual based on the biological signal. One or more health risks associated with the individual may be determined based on the health metric. Based on the one or more health risks, a treatment may be performed for the individual. In an example, the health metric may be determined (e.g., calculated) using three rules applied to one or more desaturation/resaturation events (e.g., biological events) associated with the biological signal, denoted as i. As an example, an upper threshold associated with the biological signal may be determined. As an example, the upper threshold may comprise a percentage of the biological signal. Each desaturation event (e.g., biological event) may be associated with a point/time at which the biological signal falls below the upper threshold. Each resaturation event (e.g., biological event) may be associated with a point/time at which the biological signal rises above the upper threshold. As an example, a beginning of a biological event i may be defined according to when the biological signal crosses from above to blow an upper threshold and an end of a biological event may be determined when the biological signal crosses from below to above the upper threshold. Based on Rule 1, an area under the curve (AUC) and/or an area above the curve (AAC), below the upper threshold, may be calculated for a biological event, denoted as Δᵢ. For example, if the desaturation for i starts above the upper threshold and ends below the upper threshold, any part of the area that is above the upper threshold is not included in the health metric calculation. In addition, if the resaturation for i ends above the upper threshold, any part of the area above the upper threshold is also not included in the health metric calculation. In an example, any biological event entirely above the upper threshold is not included in the health metric calculation. Based on Rule 2, any biological event where the biological signal drops below the lower threshold may be considered an artifact, and thus, may be excluded from the health metric calculation. Based on Rule 3, a weighted factor (e.g., linear weighted factor and/or non-linear weighted factor), denoted as Φᵢ, may be determined for each biological event. For each biological event, i, the AUC and/or the AAC is multiplied by its weighted factor. The health metric is calculated as the sum of all weighted biological events for an individual then multiplied by a normalization factor, denoted as Ω. As an example, the formula for determining the health metric (e.g., value or weighted hypoxemia index) may comprise Σᵢ(Δᵢ×Φᵢ)×Ω. As an example, the AUC and the AAC may be determined based on a trapezoidal rule. As an example, the weighted factor may be determined based on a linear weighted factor based on a counting technique. For example, the weighted factor may comprise a value associated with a length of a duration of a biological event. As an example, the normalization factor may be calculated as TST90c (total time in hours below 90% calculated to exclude artifacts) divided by TST (total time calculated as hours between a sleep study start and end based on manual annotation of sleep stages). As an example, the biological events may be associated with, or comprise, hypoxemia events.

In an example, the weighted factor may be determined based on a machine learning model (e.g., machine learning program/model 159). For example, the machine learning program/model 159 may include logic (e.g., hardware, software, firmware, etc.) that may be implemented to determine the weighted factor. For example, the machine learning program/model 159 may be trained based on one or more biological datasets associated with one or more biological attributes. The biological attributes may be associated with one or more of proteomics, transcriptomics, genomics, metabolomics, lipidomics, and epigenomics. For example, the biological attributes may correspond to analyses of proteins, RNA, genes, metabolites, lipids, oxidative stress measures, immune responses, and methylated DNA or modified histone proteins in chromosomes of a plurality of individuals. The trained machine learning program/model 159 may be configured to output the weighted factor based on the one or more biological datasets.

The input/output interface 160 may include an interface for delivering an instruction or data input from the individual (e.g., an operator of the computing device 101) or from a different external device (e.g., electronic device 104) to the different elements of the computing device 101. The input/output interface 160 may further include an interface for outputting one or more user interfaces to the individual. For example, the input/output interface 160 may comprise a display, such as a touch screen display, and/or one or more physical input interfaces (e.g., keyboard, mouse, etc.) configured to receive user inputs. The input/output interface 160 may be configured to output (e.g., display) a user interface comprising the health metric (e.g., weighted hypoxemia index) and the one or more health risks associated with the individual based on the health metric. In an example, the input/output interface 160 may output an instruction or data received from one or more elements of the data capture device 101 to one or more external devices (e.g., display device 102 or electronic device 104).

The communication interface 180 may establish, for example, communication between the data capture device 101 and one or more external devices (e.g., the display device 102, the electronic device 104, and/or the server 106). For example, the communication interface 180 may communicate with the one or more external devices (e.g., the display device 102, the electronic device 104, and/or the server 106) by being connected to a network 162 through wireless communication or wired communication. The network 162 may include, for example, at least one of a telecommunications network, a computer network (e.g., LAN or WAN), the Internet, and/or a telephone network.

The communication interface 180 may be configured to communicate with the one or more external devices (e.g., display device 102, or electronic device 104) via a wired communication interface 164, 165 or a wireless communication interface 164, 165. In an example, the wired communication may include, for example, at least one of Universal Serial Bus (USB), High Definition Multimedia Interface (HDMI), Recommended Standard-232 (RS-232), power-line communication, Plain Old Telephone Service (POTS), and the like. In an example, as a cellular communication protocol, the wireless communication interface 164, 165 may use at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. In an example, the wireless communication interface 164, 165 may be configured to use a near-distance communication 164, 165. The near-distance communication interface 164, 165 may include for example, at least one of Wireless Fidelity (WiFi), Bluetooth, Bluetooth Low Energy (BLE), Near Field Communication (NFC), Global Navigation Satellite System (GNSS), and the like. According to a usage region or a bandwidth or the like, the GNSS may include, for example, at least one of Global Positioning System (GPS), Global Navigation Satellite System (GLONASS), BeiDou Navigation Satellite System (BDS), Galileo, the European global satellite-based navigation system, and the like. Hereinafter, the “GPS” and the “GNSS” may be used interchangeably in the present document. In an example, the communication interface 180 may include or be communicably coupled to a transmitter, receiver and/or transceiver for communication with the external devices (e.g., display device 102, or electronic device 104).

The display device 102 may comprise one or more of a smart television, an audio/video monitor, a streaming device, and the like. The display device 102 may include various types of displays, for example, a Liquid Crystal Display (LCD) display, a Light Emitting Diode (LED) display, an Organic Light-Emitting Diode (OLED) display, a MicroElectroMechanical Systems (MEMS) display, or an electronic paper display. In an example, the display device 102 may be configured as a part of the computing device 101 or as a separate device. The display device 102 may display, for example, a variety of contents (e.g., text, image, video, icons, symbols, etc.) to the individual. For example, the display device 102 may be configured to output (e.g., display) the user interface output by the input/output interface 160. For example, the display device 102 may be configured to output (e.g., display) the health metric (e.g., weighted hypoxemia index) and the one or more health risks associated with the individual based on the health metric. For example, the computing device 101 may be configured to send the user interface to the display device 102 for the display device 102 to output to the user interface to the individual instead of, or in addition to, the computing device 101.

The electronic device 104 may comprise, for example, a laptop computer, a mobile phone, a smart phone, a tablet computer, a wearable device, a smartwatch, a haptic device, a desktop computer, a smart television, and the like. As an example, the electronic device 104 may be configured to output the user interface output by the input/output interface 160. For example, the computing device 101 may be configured to send the user interface to the electronic device 104 for the electronic device 104 to output to the user interface to the individual instead of, or in addition to, the computing device 101.

The server 106 may include a group of one or more servers. For example, all or some of the operations executed by the computing device 101 may be executed in a different one or a plurality of electronic devices (e.g., the display device 102, the electronic device 104, and/or the server 106). In an example, if the computing device 101 needs to perform a certain function or service either automatically or based on a request, the computing device 101 may request at least some parts of functions related thereto alternatively or additionally to a different electronic device (e.g., the display device 102, the electronic device 104 and/or the server 106) instead of executing the function or the service autonomously. The different electronic devices (e.g., the display device 102, the electronic device 104, or the server 106) may execute the requested function or additional function, and may deliver a result thereof to the computing device 101. The computing device 101 may provide the requested function or service either directly or by additionally processing the received result. For example, a cloud computing, distributed computing, or client-server computing technique may be used.

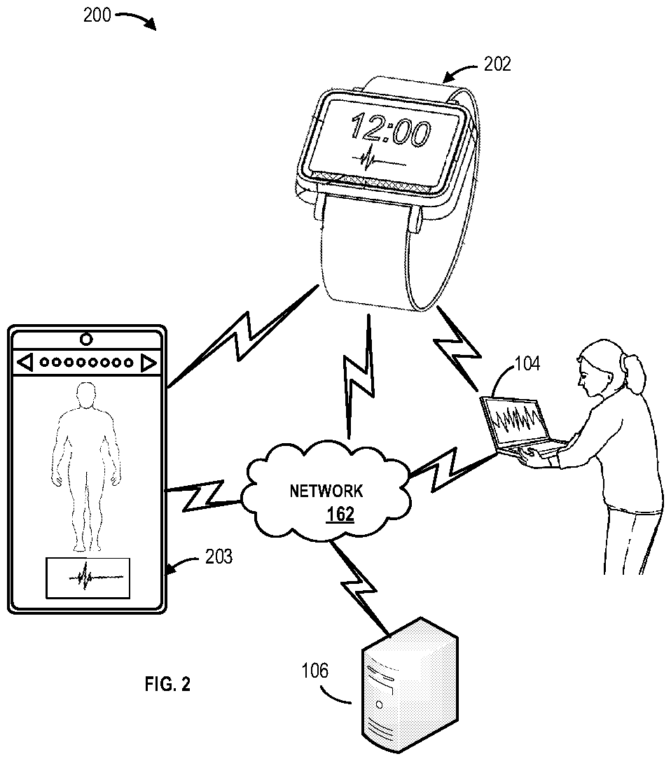

FIG. 2 shows an example system environment 200 for determining a health metric (e.g., weighted health index) and one or more health risks based on one or more biological events. For example, the system 200 may comprise an electronic device 104, a server 106, a wearable device 202, and/or a mobile device 203. The wearable device 202 (e.g., smartwatch) may be configured to receive/determine a biological signal (e.g., a pulse oximetry signal, a CO₂ signal, an arterial catheter signal (e.g., radial, carotid, or femoral), a central venous catheter signal (e.g., pulmonary artery catheter, and the like), and the like) associated with an individual. The wearable device 202 may be configured to determine a health metric (e.g., weighted hypoxemia index) associated with an individual based on the biological signal. One or more health risks associated with the individual may be determined based on the health metric. Based on the one or more health risks, a treatment may be performed for the individual. The wearable device 202 may be configured to output (e.g., display) the health metric, in addition to the one or more health risks, to the individual. In an example, the wearable device 202 may be in communication with one or more of the mobile device 203 (e.g., tablet, smartphone, etc.), the electronic device 104, and/or the sever 106 such as via a short-range connection (e.g., wired connection, Bluetooth, near-field communicate (NFC), etc.) and/or network 162. As an example, the wearable device 202 may be configured to output the health metric, in addition to the one or more health risks, to the mobile device 203 and/or the electronic device 104 for the mobile device 203 and/or the electronic device 104 to output (e.g., display) the health metric and/or the one or more health risks to the individual. As an example, the wearable device 202 may be configured to output the biological signal to the mobile device 203, the electronic device 104, and/or the server 106 for further processing. The mobile device 203, the electronic device 104, and/or the server 106 may process the biological signal to determine the health metric and/or the one or more health risks. In an example, the server 106 may store health metric data and/or health risk data associated with one or more individuals, wherein the data of each individual may be accessible by each individual and/or a medical professional.

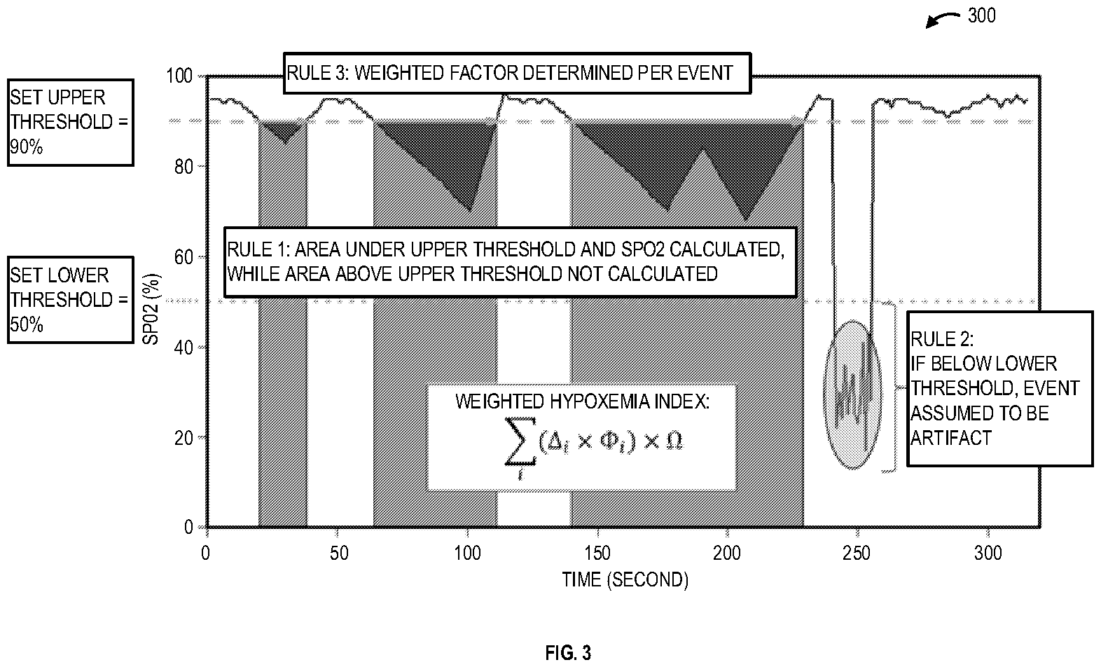

FIG. 3 shows any example, an example of a weighted hypoxemia index formulation 300. The weighted hypoxemia value may be determined based on a pulse oximetry signal. For example, pulse oximetry signal may be received via one or more of one or more sleep studies, one or more wearable devices, and/or one or more monitoring devices. The weighted hypoxemia index may be calculated using three rules applied to each desaturation/resaturation event, denoted as i. For example, a beginning of an event i may be defined according to when the pulse oximetry signal crosses from above to blow an upper threshold and an end of an event may be determined when the pulse oximetry signal crosses from below to above the upper threshold. Based on Rule 1, an area under the curve (AUC) and/or an area above the curve (AAC), below the upper threshold, may be calculated for an event, denoted as Δᵢ. Thus, if the desaturation for i starts above the upper threshold and ends below the upper threshold, any part of the area that is above the upper threshold is not included in the weighted hypoxemia index calculation. In addition, if the resaturation for i ends above the upper threshold, any part of the area above the upper threshold is also not included in the weighted hypoxemia index calculation. In an example, any event entirely above the upper threshold is not included in the weighted hypoxemia index calculation. Based on Rule 2, any event where the pulse oximetry signal drops below the lower threshold may be considered an artifact, and thus, may be excluded from the weighted hypoxemia index calculation. Based on Rule 3, a weighted factor (e.g., linear weighted factor and/or non-linear weighted factor), denoted as Φᵢ, may be determined for each event. For each event, i, the AUC or the AAC is multiplied by its weighted factor. The weighted hypoxemia index is calculated as the sum of all weighted hypoxemia events for a patient then multiplied by a normalization factor, denoted as Ω. For example, the formula for determining a weighted hypoxemia index (e.g., value) may comprise Σᵢ(Δᵢ×Φᵢ)×Ω. As an example, the upper and lower thresholds may be pre-determined based on one or more physiological attributes of an individual or one or more scenarios associated with the individual. For example, the one or more physiological attributes may comprise age, gender, hypertension, diabetes, blood pressure, oxygen saturation, pulse rate variability, or body mass index (BMI). As an example, the AUC and the AAC may be determined based on a trapezoidal rule. As an example, the weighted factor may be determined based on a linear weighted factor based on a counting technique. For example, the weighted factor may comprise a value associated with a length of duration of an event. As an example, the normalization factor may be calculated as TST90c (total time in hours below 90% calculated to exclude artifacts) divided by TST (total time calculated as hours between sleep study start and end).

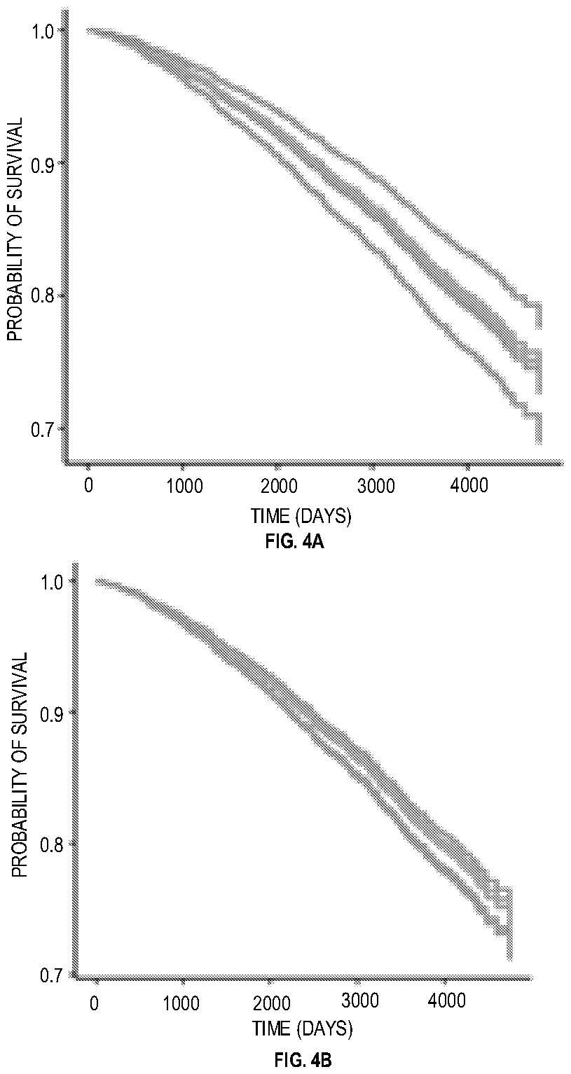

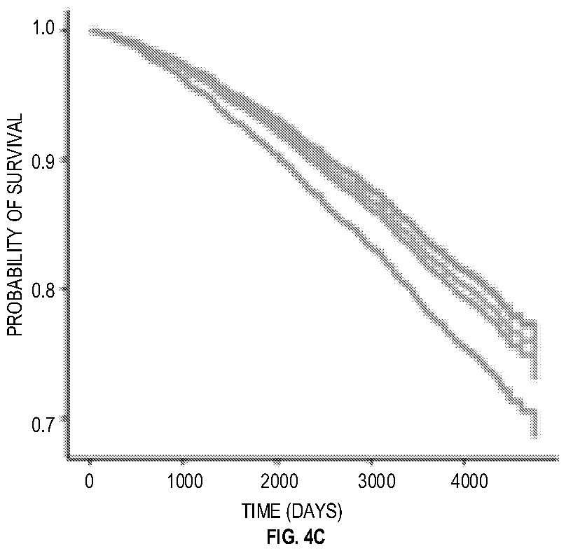

FIGS. 4A-4C show example adjusted predicted survival curves depicting all-cause mortality based on desaturations associated with poly oximetry signals associated with a plurality of individuals. The adjusted survival curves were computed by averaging the predicted survival curves for each patient within the Sleep Heart Health Study (SHHS), then averaged by a quintle. The pulse oximetry signals may be processed and analyzed to determine a severity of hypoxemia for the plurality of individuals. FIG. 4A shows adjusted curves associated with the weighted hypoxemia index. FIG. 4B shows adjusted curves associated with the apnea-hypopnea index (AHI). FIG. 4C shows adjusted curves associated with the total sleep time under oxygen saturation of 90% (TST90). As shown in Tables I-III, of the three metrics, the weighted hypoxemia index demonstrates significance across all quintiles when adjusted for demographic and cardiometabolic covariates.

TABLE I

| | WHI- | Hazard | | | |
|---|---|---|---|---|---|
| | AUC90 | Ratio | Lower CI | Upper CI | p value |
| Model 0: | Q1 | 1.00 | 1.00 | 1.00 | — |
| WHI Alone | Q2 | 1.36 | 1.10 | 1.67 | 0.0038 |
| | Q3 | 1.40 | 1.14 | 1.72 | 0.0014 |
| | Q4 | 1.62 | 1.33 | 1.98 | 0.0000 |
| | Q5 | 2.54 | 2.11 | 3.07 | 0.0000 |
| Model 1: | Q1 | 1.00 | 1.00 | 1.00 | — |
| Model 0 + | Q2 | 1.34 | 1.09 | 1.66 | 0.0056 |
| Demographic | Q3 | 1.25 | 1.02 | 1.54 | 0.0350 |
| Cardiometabolic | Q4 | 1.30 | 1.06 | 1.59 | 0.0111 |
| | Q5 | 1.64 | 1.35 | 2.00 | 0.0000 |

TABLE II

| | | Hazard | | | |
|---|---|---|---|---|---|
| | TST90 | Ratio | Lower CI | Upper CI | p value |
| Model 0: | Q1 | 1.00 | 1.00 | 1.00 | — |
| WHI Alone | Q2 | 0.87 | 0.70 | 1.08 | 0.2101 |
| | Q3 | 1.26 | 1.03 | 1.53 | 0.0224 |
| | Q4 | 1.47 | 1.21 | 1.78 | 0.0001 |
| | Q5 | 2.21 | 1.84 | 2.64 | 0.0000 |
| Model 1: | Q1 | 1.0000 | 1.00 | 1.00 | — |
| Model 0 + | Q2 | 0.99 | 0.80 | 1.22 | 0.9139 |
| Demographic | Q3 | 1.11 | 0.88 | 1.32 | 0.4557 |
| Cardiometabolic | Q4 | 1.15 | 0.95 | 1.40 | 0.1624 |
| | Q5 | 1.5215 | 1.21 | 1.78 | 0.0001 |

TABLE III

| | | Hazard | | | |
|---|---|---|---|---|---|
| | AHI | Ratio | Lower CI | Upper CI | p value |
| Model 0: | Q1 | 1.00 | 1.00 | 1.00 | — |
| WHI Alone | Q2 | 1.13 | 0.92 | 1.39 | 0.2429 |
| | Q3 | 1.33 | 1.09 | 1.62 | 0.0048 |
| | Q4 | 1.63 | 1.35 | 1.98 | 0.0000 |
| | Q5 | 1.90 | 1.57 | 2.28 | 0.0000 |
| Model 1: | Q1 | 1.00 | 1.00 | 1.00 | — |
| Model 0 + | Q2 | 0.84 | 0.68 | 1.03 | 0.0983 |
| Demographic | Q3 | 0.91 | 0.74 | 1.11 | 0.3596 |
| Cardiometabolic | Q4 | 0.88 | 0.72 | 1.07 | 0.2048 |
| | Q5 | 1.02 | 0.84 | 1.25 | 0.8147 |

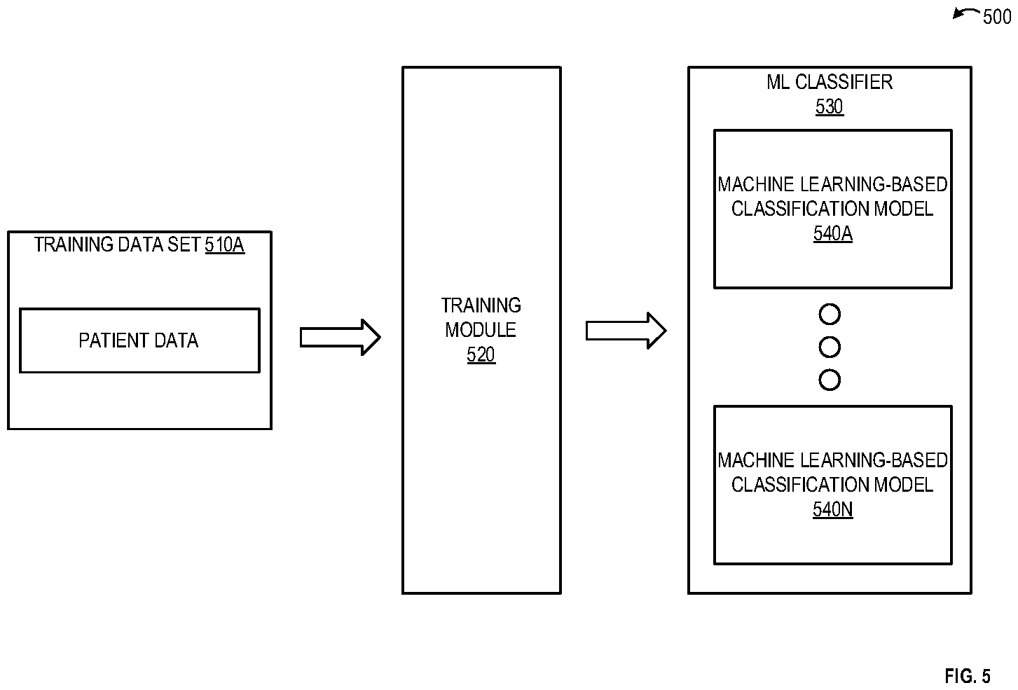

FIG. 5 shows a system 500 that is configured to use machine learning techniques to train, based on an analysis of one or more training datasets 510 by a training module 520, at least one machine learning-based classifier 530 that is configured to classify expected one or more biological attributes based on one or more biological datasets to determine a weighted factor associated with a biological event of an individual. For example, the one or more biological attributes may be associated with one or more of proteomics, transcriptomics, genomics, metabolomics, lipidomics, and epigenomics. For example, the biological attributes may correspond to analyses of proteins, RNA, genes, metabolites, lipids, and methylated DNA or modified histone proteins in chromosomes of a plurality of individuals. As an example, the training datasets 510 (e.g., the biological datasets) may comprise one or more groups of biological features and/or baseline feature levels. As an example, the training dataset 510 may comprise labeled baseline feature levels (e.g., baseline feature scores). The labels may comprise a plurality of predefined features associated with the one or more groups of biological attributes.

The training module 520 may train the machine learning-based classifier 530 by extracting a feature set from the biological datasets (e.g., one or more groups of biological attributes and/or baseline feature levels) in the training data set 510 according to one or more feature selection techniques.

In an example, the training module 520 may extract a feature set from the training dataset 510 in a variety of ways. The training module 520 may perform feature extraction multiple times, each time using a different feature-extraction technique. In an embodiment, the feature sets generated using the different techniques may each be used to generate different machine learning-based classification models 540. As an example, the feature set with the highest quality metrics may be selected for use in training. The training module 520 may use the feature set(s) to build one or more machine learning-based classification models 540A-540N that are configured to indicate whether or not new data is associated with weighted factor.

In an example, the training data set 510 may be analyzed to determine one or more groups of biological attributes that have at least one feature that may be used to determine a weighted factor associated with a biological event. The one or more groups of biological attributes may be considered as features (or variables) in the machine learning context. The term “feature,” as used herein, may refer to any characteristic of a group biological attributes that may be used to determine a weighted factor associated with a biological event.

In an example, a feature selection technique may comprise one or more feature selection rules. The one or more feature selection rules may comprise a biological attribute occurrence rule. The biological attribute occurrence rule may comprise determining which biological attributes, or group of biological attributes, in the training data set 510 occur over a threshold number of times and identifying those biological attributes that satisfy the threshold as candidate features. For example, any biological attribute, or group of biological attributes, that appear greater than or equal to 50 times in the training data set 510 may be considered as candidate features. Any biological attribute, or group of biological attributes, appearing less than 50 times may be excluded from consideration as a feature.

In an example, the one or more feature selection rules may comprise a significance rule. The significance rule may comprise determining, from the baseline feature level (e.g., baseline feature score) data in the training data set 510, biological attribute feature data. The biological attribute feature data be associated with one or more of proteomics, transcriptomics, genomics, metabolomics, lipidomics, and epigenomics. For example, the biological attributes may correspond to analyses of proteins, RNA, genes, metabolites, lipids, and methylated DNA or modified histone proteins in chromosomes of a plurality of individuals. As the baseline feature level (e.g., baseline feature score) in the training data set 510 are labeled according to one or more biological attribute features, the labels may be used to determine the weighted factor associated with a biological event.

In an example, a single feature selection rule may be applied to select features or multiple feature selection rules may be applied to select the features. For example, the feature selection rules may be applied in a cascading fashion, with the feature selection rules being applied in a specific order and applied to the results of the previous rule. For example, the biological attribute occurrence rule may be applied to the training data set 510 to generate a first list of features. The significance rule may be applied to features in the first list of features to determine which features of the first list satisfy the significance rule in the training data set 510 and to generate a final list of candidate features.

The final list of candidate features may be analyzed according to additional feature selection techniques to determine one or more candidate feature signatures (e.g., groups biological attributes). Any suitable computational technique may be used to identify the candidate feature signatures using any feature selection technique such as filter, wrapper, and/or embedded methods. In an example, one or more candidate feature signatures may be selected according to a filter method. Filter methods include, for example, Pearson's correlation, linear discriminant analysis, analysis of variance (ANOVA), chi-square, combinations thereof, and the like. The selection of features according to filter methods are independent of any machine learning algorithms. Instead, features may be selected on the basis of scores in various statistical tests for their correlation with the outcome variable (e.g., an expected weighted factor result).

In an example, one or more candidate feature signatures may be selected according to a wrapper method. A wrapper method may be configured to use a subset of features and train a machine learning model using the subset of features. Based on the inferences that are drawn from a previous model, features may be added and/or deleted from the subset. Wrapper methods include, for example, forward feature selection, backward feature elimination, recursive feature elimination, combinations thereof, and the like. As an example, forward feature selection may be used to identify one or more candidate feature signatures. Forward feature selection is an iterative method that begins with no feature in the machine learning model. In each iteration, the feature which best improves the model is added until an addition of a new variable does not improve the performance of the machine learning model. As an example, backward elimination may be used to identify one or more candidate feature signatures. Backward elimination is an iterative method that begins with all features in the machine learning model. In each iteration, the least significant feature is removed until no improvement is observed on removal of features. As an example, recursive feature elimination may be used to identify one or more candidate feature signatures. Recursive feature elimination is a greedy optimization algorithm which aims to find the best performing feature subset. Recursive feature elimination repeatedly creates models and keeps aside the best or the worst performing feature at each iteration. Recursive feature elimination constructs the next model with the features remaining until all the features are exhausted. Recursive feature elimination then ranks the features based on the order of their elimination.

In an example, one or more candidate feature signatures may be selected according to an embedded method. Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs L1 regularization which adds a penalty equivalent to the absolute value of the magnitude of coefficients and ridge regression performs L2 regularization which adds a penalty equivalent to the square of the magnitude of coefficients.

After the training module 520 has generated a feature set(s), the training module 520 may generate a machine learning-based classification model 540 based on the feature set(s). The machine learning-based classification model 540, may refer to a complex mathematical model for data classification that is generated using machine-learning techniques. In an example, this machine learning-based classifier may include a map of support vectors that represent boundary features. For example, boundary features may be selected from, and/or represent the highest-ranked features in, a feature set.

In an example, the training module 520 may use the feature sets extracted from the training data set 510 to build a machine learning-based classification model 540A-540N for each classification category (e.g., biological attribute classification). In an example, the machine learning-based classification models 540A-540N may be combined into a single machine learning-based classification model 540. Similarly, the machine learning-based classifier 530 may represent a single classifier containing a single or a plurality of machine learning-based classification models 540 and/or multiple classifiers containing a single or a plurality of machine learning-based classification models 540.

The extracted features (e.g., one or more candidate features and/or candidate feature signatures derived from the final list of candidate features) may be combined in a classification model trained using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting machine learning-based classifier 530 may comprise a decision rule or a mapping that uses the expression levels of the features in the candidate feature signature to determine a weighted factor associated with a hypoxemia event.

The candidate feature signature and the machine learning-based classifier 530 may be used to determine a weighted factor associated with a hypoxemia event in the testing data set. In an example, the result for each test includes a confidence level that corresponds to a likelihood or a probability that the corresponding test predicted a weighted factor result. The confidence level may be a value between zero and one that represents a likelihood that the corresponding test is associated with a weighted factor result. In one example, when there are two or more statuses (e.g., two or more expected weighted factor results), the confidence level may correspond to a value p, which refers to a likelihood that a particular test is associated with a first status. In this case, the value 1−p may refer to a likelihood that the particular test is associated with a second status. In general, multiple confidence levels may be provided for each test and for each candidate feature signature when there are more than two statuses. A top performing candidate feature signature may be determined by comparing the result obtained for each test with known expected weighted factor results for each test. In general, the top performing candidate feature signature will have results that closely match the known weighted factors.

The top performing candidate feature signature may be used to predict the expected weighted factor result. For example, patient datasets and/or baseline feature data may be determined/received. The patient datasets and/or the baseline feature data may be provided to the machine learning-based classifier 530 which may, based on the top performing candidate feature signature, predict/determine an expected weighted factor result. Based on the predicted weighted factor, a health metric (e.g., weighted hypoxemia index) may be determined.

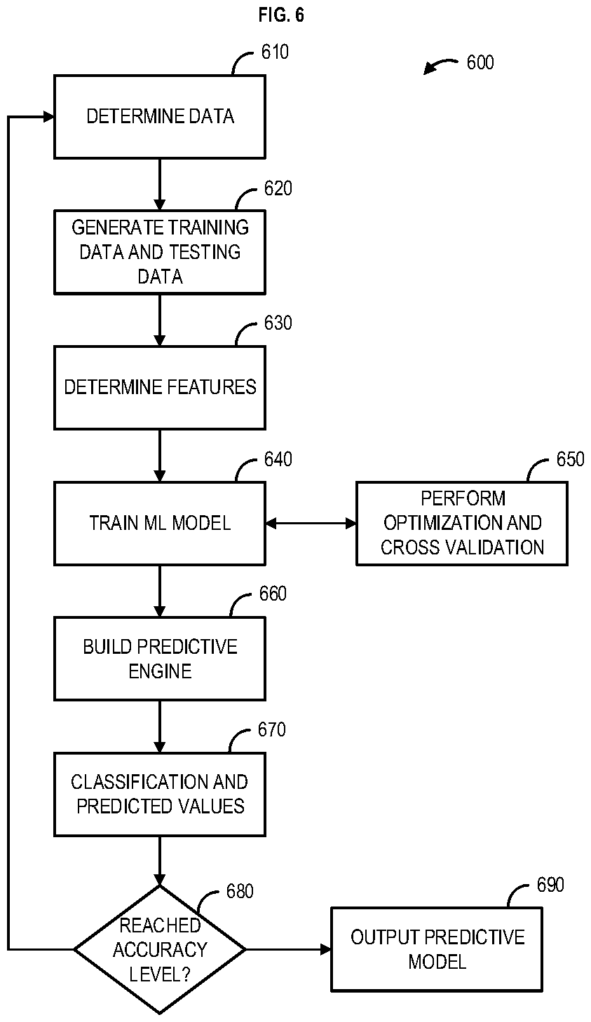

FIG. 6 shows a flowchart of an example training method 600 for generating the machine learning-based classifier 530 using the training module 520. The training module 520 may be implement using supervised, unsupervised, and/or semi-supervised (e.g., reinforcement based) machine learning-based classification models 540. The method 600 illustrated in FIG. 6 is an example of a supervised learning method; variations of this example of training method are discussed below, however, other training methods may be analogously implemented to train unsupervised and/or semi-supervised machine learning models.

The training method 600 may determine (e.g., access, receive, retrieve, etc.) one or more biological datasets associated with one or more biological attributes at 610. For example, the one or more biological attributes may be associated with one or more of proteomics, transcriptomics, genomics, metabolomics, lipidomics, and epigenomics. For example, the biological attributes may correspond to analyses of proteins, RNA, genes, metabolites, lipids, and methylated DNA or modified histone proteins in chromosomes of a plurality of individuals.

The training method 600 may generate, at 620, a training data set and a testing data set. The training data set and the testing data set may be generated by randomly assigning labeled feature data of individual features from the biological datasets to either the training dataset or the testing dataset. In an example, the assignment of the labeled feature data of individual features may not be completely random. In an example, only the labeled feature data for a group of biological attributes may be used to generate the training dataset and the testing dataset. In an example, a majority of the labeled feature data for the specific study may be used to generate the training dataset. For example, 75% of the labeled feature data for the specific study may be used to generate the training dataset and 25% may be used to generate the testing dataset. In an example, only the labeled feature data for the specific study may be used to generate the training dataset and the testing dataset.

The training method 600 may determine (e.g., extract, select, etc.), at 630, one or more features that can be used by, for example, a classifier to differentiate among different classifications (e.g., different groups of biological attributes). The one or more features may comprise a group of biological attributes. In an example, the training method 600 may determine a set of features from the biological datasets. In an example, a set of features may be determined from a different group of biological attributes (e.g., second group of biological attributes) associated with the labeled feature data of the training data set and the testing data set. In other words, the second group of biological attributes may be used for feature determination, rather than for training a machine learning model. In an example, the training data set may be used in conjunction with the second group of biological attributes to determine the one or more features. The patient data from the second group of biological attributes may be used to determine an initial set of features, which may be further reduced using the training data set.

The training method 600 may train one or more machine learning models using the one or more features at 640. As an example, the machine learning models may be trained using supervised learning. As an example, other machine learning techniques may be employed, including unsupervised learning and semi-supervised. The machine learning models trained at 640 may be selected based on different criteria depending on the problem to be solved and/or data available in the training data set. For example, machine learning classifiers can suffer from different degrees of bias. Accordingly, more than one machine learning model may be trained at 640, optimized, improved, and cross-validated at 650.

The training method 600 may select one or more machine learning models to build a predictive model at 660 (e.g., a machine learning classifier). The predictive model may be evaluated using the testing dataset. The predictive model may analyze the testing data set and generate classification values and/or predicted values at 670. Classification and/or prediction values may be evaluated at 680 to determine whether such values have achieved a desired accuracy level. Performance of the predictive model may be evaluated in a number of ways based on a number of true positive, false positive, true negative, and/or false negative classifications of the plurality of data points indicated by the predictive model. For example, the false positives of the predictive model may refer to a number of times the predictive model incorrectly classified a patient's weighted factor based on the biological datasets. Conversely, the false negatives of the predictive model may refer to a number of times the machine learning model determined that a weighted factor was not associated with a biological dataset when, in fact, the weighted factor was associated with the biological dataset. True negatives and true positives may refer to a number of times the predictive model correctly classified a weighted factor based on a biological dataset. Related to these measurements are the concepts of recall and precision. Generally, recall refers to a ratio of true positives to a sum of true positives and false negatives, which quantifies a sensitivity of the predictive model. Similarly, precision refers to a ratio of true positives a sum of true and false positives.

When a desired accuracy level is reached, the training phase ends and the predictive model may be output at 690; when the desired accuracy level is not reached, however, then a subsequent iteration of the training method 600 may be performed starting at 610 with variations such as, for example, considering a larger collection of biological datasets.

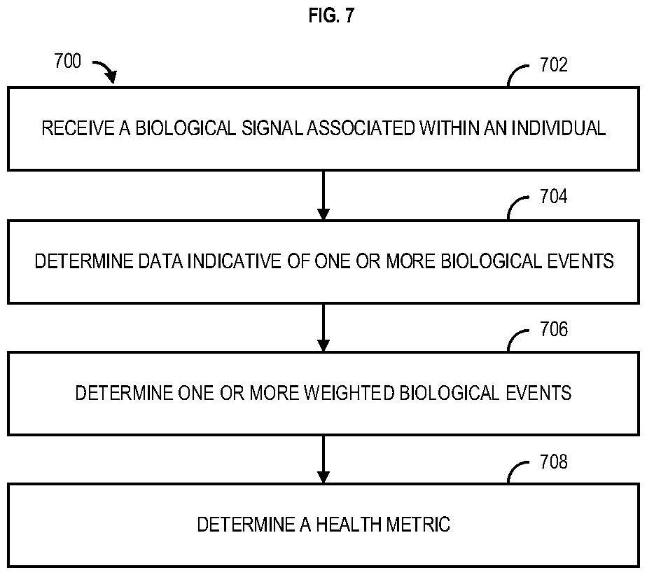

FIG. 7 shows a flowchart of an example method 700. The method 700 may be implemented in whole, or in part, by a computing device (e.g., computing device 101, electronic device 104, and/or server 106). At step 702, a biological signal associated with an individual may be received. For example, the biological signal may be received by the computing device (e.g., computing device 101, electronic device 104, and/or server 106). In an example, the biological signal comprise one or more of a pulse oximetry signal, a CO₂ signal, an arterial catheter signal (e.g., radial, carotid, or femoral), a central venous catheter signal (e.g., pulmonary artery catheter, and the like), and the like.

At step 704, data indicative of a one or more biological events may be determined based on the biological signal. For example, the data indicative of the one or more biological events may be determined by the computing device (e.g., computing device 101, electronic device 104, and/or server 106) based on the biological signal. As an example, the biological event may be associated with, or comprise, a hypoxemia event. Each biological event of the one or more biological events may be associated with a weighted factor. The weighted factor may comprise one or more of a linear value or a non-linear value. As an example, the weighted factor may be generated based on a machine learning model. The machine learning model may be trained based on one or more biological datasets associated with one or more biological attributes associated with one or more individuals. The biological attributes may be associated with one or more of proteomics, transcriptomics, genomics, metabolomics, lipidomics, and epigenomics. For example, the biological attributes may correspond to analyses of proteins, RNA, genes, metabolites, lipids, oxidative stress measures, immune responses, and methylated DNA or modified histone proteins in chromosomes of one or more individuals.

In an example, a beginning of each biological event may be determined based on a point at which the biological signal crosses from above a threshold (e.g., upper threshold) to below the threshold and an end of each biological event may be determined based on a point at which the biological signal crosses from below the threshold to above the threshold. The data indicative of the one or more biological events may be determined based on the beginning of each biological event and the end of each biological event. As an example, the threshold (e.g., upper threshold) may be a predetermined threshold comprising a percentage of the biological signal.

At step 706, one or more weighted biological events may be determined based on an application of each weighted factor to each biological event. For example, the one or more weighted biological events may be determined by the computing device (e.g., computing device 101, electronic device 104, and/or server 106) based on an application of each weighted factor to each biological event. In an example, for each biological event, one or more of an area under the biological signal or an area above the biological signal below the threshold may be determined. The one or more weighted biological events may be determined based on multiplying the weighted factor of each biological event with one or more of the area under the biological signal or the area above the biological signal below the threshold. In an example, it may be determined that at least one biological event is associated with a point at which the biological signal is below a threshold (e.g., lower threshold). The data indicative the at least one biological event may be excluded from the data indicative of the one or more biological events. As an example, the threshold (e.g., lower threshold) may be a predetermined threshold comprising a percentage of the biological signal.

At step 708, a health metric (e.g., a weighted hypoxemia index) may be determined based on the one or more weighted biological events. For example, the health metric may be determined by the computing device (e.g., computing device 101, electronic device 104, and/or server 106) based on the one or more weighted biological events. As an example, a summation of the one or more weighted biological events may be determined. The health metric may be determined based on an application of a normalization factor to the summation of the one or more weighted biological events. In an example, one or more health risks associated with the individual may be determined based on the health risk. The individual may be provided a treatment based on the one or more health risks. As an example, the one or more health risks may comprise one or more of heart disease, cancer, COVID-19, accidental injuries, stroke, chronic lower respiratory diseases, Alzheimer's disease, diabetes, influenza, pneumonia, and/or kidney disease.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method comprising:
receiving, by a computing device, a biological signal associated with an individual;
determining, based on the biological signal, data indicative of one or more biological events, wherein each biological event of the one or more biological events is associated with a weighted factor;
determining, based on an application of each weighted factor to each biological event, one or more weighted biological events; and
determining, based on the one or more weighted biological events, a health metric.

2. The method of claim 1, wherein the biological signal comprises one or more of a pulse oximetry signal, a CO₂ signal, an arterial catheter signal, or a central venous catheter signal.

3. The method of claim 1, wherein the biological event is associated with a hypoxemia event.

4. The method of claim 1, wherein determining, based on the biological signal, the data indicative of one or more biological events comprises:
determining, based on a point at which the biological signal crosses from above a threshold to below the threshold, a beginning of each biological event;
determining, based on a point at which the biological signal crosses from below the threshold to above the threshold, an end of each biological event; and
determining, based on the beginning of each biological event and the end of each biological event, the data indicative of one or more biological events.

5. The method of claim 4, further comprises determining, for each biological event, one or more of an area under the biological signal or an area above the biological signal below the threshold, wherein determining, based on the application of each weighted factor to each biological event, the one or more weighted biological events comprises determining, based on multiplying the weighted factor of each biological event with one or more of the area under the biological signal or the area above the biological signal below the threshold, the one or more weighted biological events.

6. The method of claim 1, further comprising:
determining at least one biological event associated with a point at which the biological signal is below a threshold; and
excluding data indicative the at least one biological event from the data indicative of the one or more biological events.

7. The method of claim 1, wherein the weighted factor comprises one or more of a linear value or a non-linear value.

8. The method of claim 1, further comprising generating the weighted factor based on a machine learning model, wherein the machine learning model is trained based on one or more biological datasets associated with one or more biological attributes.

9. The method of claim 1, wherein determining, based on the one or more weighted biological events, the health metric comprises:
determining a summation of the one or more weighted biological events; and
determining, based on an application of a normalization factor to the summation of the one or more weighted biological events, the health metric.

10. The method of claim 1, further comprising:
determining, based on the health metric, one or more health risks associated with the individual; and
causing, based on the one or more health risks, a treatment of the individual.

11. An apparatus comprising:
one or more processors; and
a memory storing processor-executable instructions that, when executed by the one or more processors, cause the apparatus to:
receive a biological signal associated with an individual;
determine, based on the biological signal, data indicative of one or more biological events, wherein each biological event of the one or more biological events is associated with a weighted factor;
determine, based on an application of each weighted factor to each biological event, one or more weighted biological events; and
determine, based on the one or more weighted biological events, a health metric.

12. The apparatus of claim 11, wherein the biological signal comprises one or more of a pulse oximetry signal, a CO₂ signal, an arterial catheter signal, or a central venous catheter signal.

13. The apparatus of claim 11, wherein the biological event is associated with a hypoxemia event.

14. The apparatus of claim 11, wherein the processor-executable instructions that, when executed by the one or more processors, cause the apparatus to determine, based on the biological signal, the data indicative of one or more biological events further cause the apparatus to:
determine, based on a point at which the biological signal crosses from above a threshold to below the threshold, a beginning of each biological event;
determine, based on a point at which the biological signal crosses from below the threshold to above the threshold, an end of each biological event; and
determine, based on the beginning of each biological event and the end of each biological event, the data indicative of one or more biological events.

15. The apparatus of claim 14, wherein the processor-executable instructions, when executed by the one or more processors, further cause the apparatus to determine, for each event, one or more of an area under the biological signal or an area above the biological signal below the threshold, wherein the processor-executable instructions that, when executed by the one or more processors, cause the apparatus to determine, based on the application of each weighted factor to each biological event, the one or more weighted biological events, further cause the apparatus to determine, based on multiplying the weighted factor of each biological event with one or more of the area under the biological signal or the area above the biological signal below the threshold, the one or more weighted biological events.

16. The apparatus of claim 11, wherein the processor-executable instructions, when executed by the one or more processors, further cause the apparatus to:
determine at least one biological event associated with a point at which the biological signal is below a threshold; and
exclude data indicative of the at least one biological event from the data indicative of the one or more biological events.

17. The apparatus of claim 11, wherein the weighted factor comprises one or more of a linear value or a non-linear value.

18. The apparatus of claim 11, wherein the processor-executable instructions, when executed by the one or more processors, further cause the apparatus to generate the weighted factor based on a machine learning model, wherein the machine learning model is trained based on one or more patient datasets associated with one or more biological attributes.

19. The apparatus of claim 11, wherein the processor-executable instructions that, when executed by the one or more processors, cause the apparatus to determine based on the one or more weighted biological events, the health metric, further cause the apparatus to:
determine a summation of the one or more weighted biological events; and
determine, based on an application of a normalization factor to the summation of the one or more weighted biological events, the health metric.

20. The apparatus of claim 11, wherein the processor-executable instructions, when executed by the one or more processors, further cause the apparatus to:
determine, based on the health metric, one or more health risks associated with the individual; and
causing, based on the one or more health risks, a treatment of the individual.
